(19) United States
(12) Patent Application Publication
(10) Pub. No.: US 2009/0118575 A1
(43) Pub. Date: May 7, 2009

(54) ENDOSCOPIC SYSTEM, TREATMENT SECTION OPERATION CHECK INSTRUMENT FOR THE SAME, AND TREATMENT SECTION OPERATION CHECK METHOD

(75) Inventors: Hiroaki Ichikawa, Tokyo (JP); Kazushi Murakami, Tokyo (JP); Takaaki Komiya, Tokyo (JP); Tsutomu Okada, Tokyo (JP); Yoshio Onuki, Tokyo (JP); Yasuhito Kura, Tokyo (JP); Kazuki Honda, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 11/935,634

(22) Filed: Nov. 6, 2007

(51) Int. Cl. A61B 1/005

(52) U.S. Cl. 600/103; 600/106

(57) ABSTRACT

An endoscopic system includes an endoscope, a cartridge, and a treatment section operation check instrument. The endoscope includes a treatment instrument channel. The cartridge stores a treatment section and a treatment instrument insertion portion introduced into the treatment instrument channel. The treatment section operation check instrument includes a chamber. The chamber contains a hollow section with the volume which allows the treatment section stored in the cartridge to perform a treatment operation. The treatment section operation check instrument is disposed between the endoscope and the cartridge so as to be allowed to perform the operation check by disposing the treatment section in the hollow section of the chamber before insertion of the treatment section into the treatment instrument channel.

### BACKGROUND OF THE INVENTION

1. Field of the Invention

The present invention relates to an endoscopic system including an endoscope inserted into a body cavity, and a cartridge which stores various types of treatment instruments used together with the endoscope, which conducts an operation check whether or not the treatment section of the treatment instrument stored in the cartridge performs a predetermined operation, a treatment section operation check instrument provided in the endoscopic system, which includes a hollow section between the endoscope and the cartridge for conducting an operation check, and an operation check method for checking whether or not the treatment section of the treatment instrument performs a predetermined operation.

2. Description of the Related Art

Generally, an endoscope has been widely employed in the industrial field or the medical field. In the medical field, the endoscope is inserted into the body cavity of the subject body for the purpose of observation. The treatment instrument is introduced into the body cavity via a treatment instrument channel installed in the insertion portion of the endoscope to perform the biopsy or various kinds of treatment.

The operator introduces the treatment instrument into the body cavity via the treatment instrument cannel of the endoscope for the purpose of taking the body cavity tissue or performing the treatment.

The operator introduces the treatment instrument into the body cavity while grasping the operation portion of the endoscope with one hand. The operator then holds the sheath as the insertion portion of the treatment instrument with the other hand to manually insert the sheath into the treatment instrument channel. The proximal end of the sheath is held by the nursing staff for the purpose of preventing a part of the sheath with the length of, for example, 2 meters from being in contact with the uncleaned area such as floor during the insertion.

Meanwhile, the operator takes the body tissue with the treatment instrument while grasping the operation portion of the endoscope with one hand. It is therefore impossible for the operator to hold the insertion portion of the endoscope and operate the operation portion of the treatment instrument with the other hand. Accordingly, the staff is expected to hold the insertion portion of the endoscope or to operate the operation portion of the treatment instrument.

The assistance of the staff is required for inserting the sheath of the treatment instrument into the treatment instrument channel of the endoscope, or operating the treatment instrument which has been inserted into the treatment instrument channel.

Japanese Unexamined Patent Application Publication No. 2005-152502 (referred to as Patent Document 1) discloses the endoscopic treatment instrument insertion system equipped with the treatment instrument unit including the biopsy forceps with an insertion portion which can be inserted into or pulled out from the forceps channel. According to the aforementioned disclosure, the operator does not have to support the treatment instrument upon insertion/pull-out of the treatment instrument.

Japanese Unexamined Patent Application Publication No. 2006-25823 (referred to as Patent Document 2) discloses the endoscopic treatment system provided with a rotatable reel in the endoscope for winding the insertion portion of the treatment instrument inserted into the treatment instrument channel of the endoscope, and the endoscopic treatment instrument. According to the disclosure, the treatment instrument may be easily operated with the simple structure. Furthermore, the operator of the endoscope is allowed to operate the treatment instrument.

In the structure of Patent Document 1, after setting the treatment instrument unit in the endoscope, the treatment section of the biopsy forceps cannot be opened/closed until the treatment section protrudes from the distal end of the endoscope. In the structure disclosed in Patent Document 2, the treatment section of the treatment instrument cannot be driven until the treatment section protrudes from the distal end of the endoscope. The confirmation whether or not the treatment section is driven cannot be made until actual operation of the treatment section.

### SUMMARY OF THE INVENTION

The endoscopic system according to the present invention includes an endoscope, a cartridge, and a treatment section operation check instrument. The endoscope includes the treatment instrument channel. The cartridge stores the treatment section and the treatment instrument insertion portion introduced into the treatment instrument channel. The treatment section operation check instrument includes the chamber. The changer further includes a hollow section with the volume which allows the treatment section stored in the cartridge to perform a treatment operation. The treatment section operation check instrument with the chamber is disposed between the endoscope and the cartridge for checking the operation by arranging the treatment section in the hollow section of the chamber before insertion of the treatment section into the treatment instrument channel.

The treatment section operation check instrument according to the present invention includes the chamber and a pair of communication portions. The chamber includes the hollow section where the treatment section of the endoscopic treatment instrument stored in the sheath storage portion of the cartridge is disposed. The hollow section has the volume which allows the operation check of the treatment section. The chamber includes the transparent portion which allows the treatment section in the hollow section to be visually recognized. The communication portion is communicated with the hollow section of the chamber The longitudinal shaft of one of the communication portions is coaxial with the longitudinal shaft of the other communication portion.

The above and other objects, features and advantages of the invention will become more clearly understood from the following description referring to the accompanying drawings.

### BRIEF DESCRIPTION OF THE DRAWINGS

FIG. 1 is a perspective view showing an endoscopic system where a treatment section operation check instrument is disposed between an endoscope and a cartridge.

FIG. 2 is an explanatory view including a block diagram of the endoscopic system having a treatment section operation check instrument disposed between the endoscope and the cartridge.

FIG. 3 is an explanatory view showing a relationship between a tilting operation of an operation lever of a controller and an operation of the treatment section of the treatment instrument.

FIG. 4 is a view showing a relationship between the tilting operation of a rotary lever of the controller and operations of the treatment section of the treatment instrument and the sheath.

FIG. 5 is a view showing the treatment section operation check instrument.

FIG. 6 is a view showing the treatment section operation check instrument which includes a flexible tube formed by integrating the outlet and the connecting tube.

FIG. 7 is a view showing the endoscopic system including the treatment section operation check instrument with the flexible tube formed by integrating the inlet and the connecting tube.

FIG. 8 is a view showing the treatment section operation check instrument including a lens chamber provided with a lens portion.

FIG. 9 is an explanatory view showing an example of a marking applied on the treatment section, and a function of the marking.

FIG. 10 is an explanatory view showing another example of the marking applied on the treatment section, and the function of the marking.

FIG. 11 is an explanatory view showing the operation check performed by protruding the treatment section from the opening of the distal end portion of the convex portion of an electric driver.

FIG. 12 is an explanatory view showing an endoscopic system provided with an endoscope, a cartridge, a control unit which contains the treatment section operation check program in the control section, the treatment instrument insertion portion electric driver, the treatment section operation unit, the treatment section operation unit including the operation check command button for outputting the command signal to perform the operation check based on the treatment section operation check program to the control unit, and the treatment section operation check instrument including the detection unit disposed between the endoscope and the cartridge.

FIG. 13 is a flowchart showing the routine for executing the operation check based on the treatment section operation check program in the endoscopic system shown in FIG. 12.

FIG. 14 is an explanatory view showing the subscreen which notifies the operator of the next operation, and a display unit provided with a buzzer.

FIG. 15 is an explanatory view showing the treatment section operation check instrument including the chamber with the shape other than the spherical shape.

FIG. 16 is an explanatory view showing the structure and function of the treatment section operation check instrument having a retractable shutter member with respect to the hollow section, which is provided in the chamber.

FIG. 17 is an explanatory view showing the structure in which an observation camera as the inspection portion for observing the inside of the hollow section is provided in the chamber of the treatment section operation check instrument, and the function thereof.

FIG. 18 is an explanatory view showing the endoscope equipped with a sensor for detecting the approach of the treatment section to the vicinity of the treatment instrument insertion port of the treatment instrument channel.

FIG. 19 is an explanatory view showing the function of the endoscope equipped with the sensor adjacent to the treatment instrument insertion port.

FIG. 20 is an explanatory view showing the endoscopic system equipped with the sensor for detecting the approach of the treatment section to the vicinity of the connecting adaptor of the treatment instrument insertion port.

FIG. 21 is an explanatory view showing the function of the endoscopic system having the sensor installed in the connecting adaptor.

FIG. 22 is an explanatory view showing a cartridge with chamber including a pair of pulleys, a drum, and a drive belt in a main body which includes a chamber portion having a hollow section and a cartridge portion including the sheath storage portion.

FIG. 23 is a cross section taken on line XXIII-XXIII shown in FIG. 22, mainly showing the structure of the inside of the main body.

FIG. 24 is an explanatory view showing the function of the cartridge with chamber.

### DETAILED DESCRIPTION OF THE PREFERRED EMBODIMENTS

Embodiments of the present invention will be described referring to the drawings.

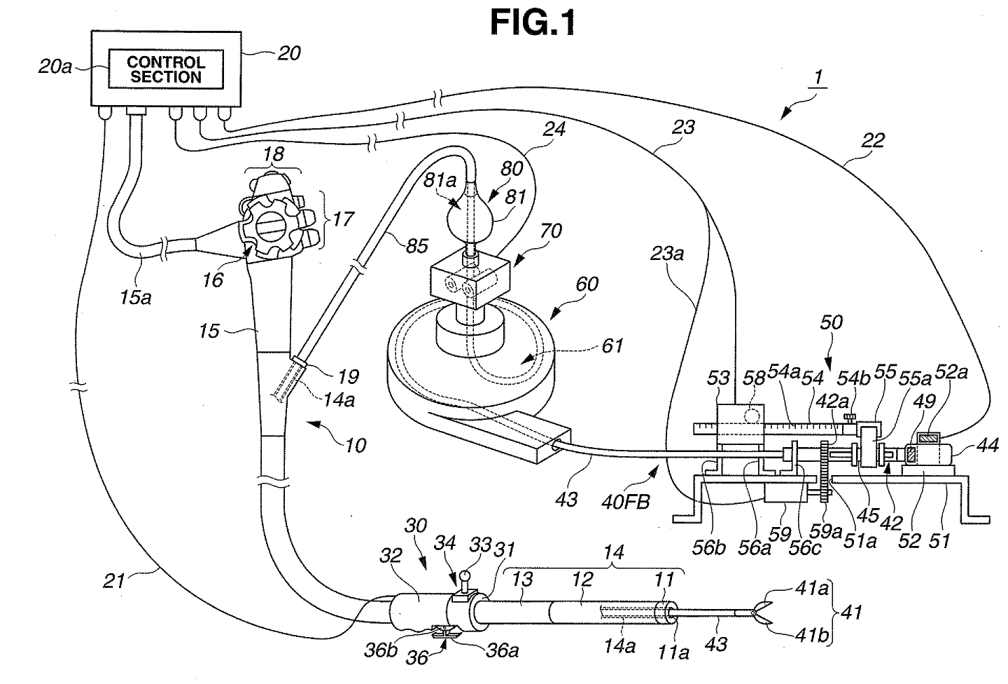

A first embodiment of the present invention will be described referring to FIGS. 1 to 11.

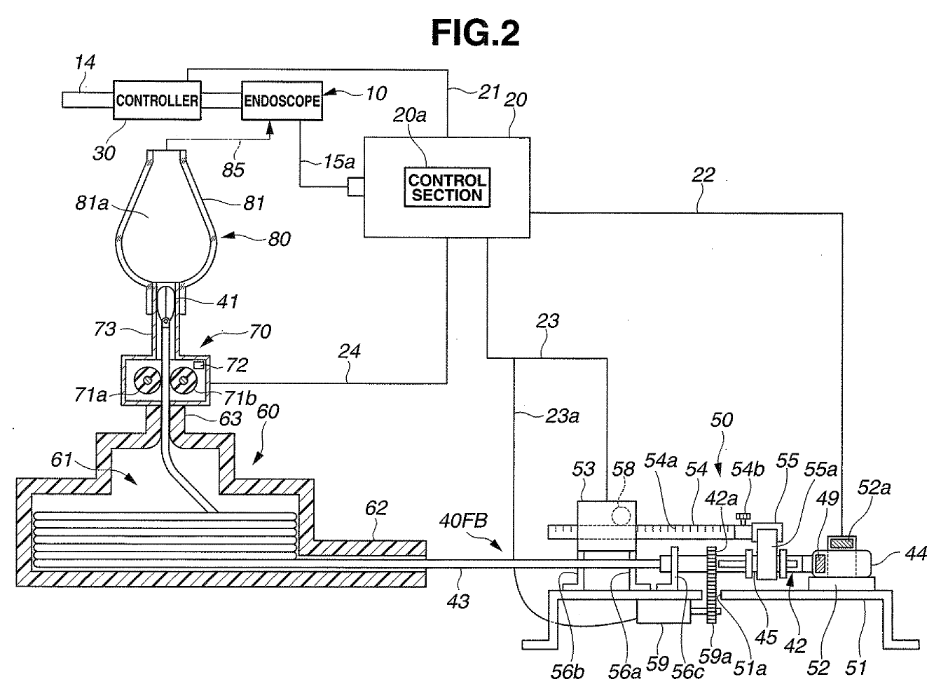

Referring to FIGS. 1 and 2, an endoscopic system 1 as a medical system according to the embodiment mainly includes an endoscope 10, a control unit 20, a controller 30, an endoscopic treatment instrument (hereinafter referred to as a treatment instrument) 40, a treatment instrument electric opening/closing rotary device (hereinafter referred to as an electric opening/closing rotary device) 50, a storage case hereinafter referred to as a cartridge) 60, a treatment instrument insertion portion electric driver (hereinafter referred to as an electric driver) 70 and a treatment section operation check instrument 80. The control unit 20 includes a control section 20a. The controller 30 serves as an operation command device for outputting the command signal to the electric opening/closing rotary device 50 and the electric driver 70. The treatment instrument 40 includes a treatment section 41 for performing a predetermined treatment to the body cavity tissue, and a handle 42 which allows the treatment section 41 to operate. The electric opening/closing rotary device 50 serves as the treatment section operation device for operating the treatment section. The treatment section 41 is operated by electrically operating the handle 42 set in the electric opening/closing rotary device 50. The cartridge 60 includes a sheath storage portion 61 for storing the treatment section 41 of the treatment instrument 40, and a sheath 43 as the treatment instrument insertion portion of the treatment instrument 40 to be wound therein. The electric driver 70 serves as the treatment instrument driver which electrically drives the sheath 43 of the treatment instrument 40 forward and backward. The treatment section operation check instrument 80 includes a chamber 81 with a check hollow section 81a (hereinafter referred to as the hollow section), in which the treatment section 41 of the treatment instrument 40 is disposed.

The endoscope 10 includes an endoscopic insertion portion (hereinafter referred to as an insertion portion) 14 on which a distal end portion 11, a bending portion 12, and a flexible tube 13 are connected in the order from the distal end side. The distal end portion 11 formed of a rigid member has a distal end opening 11a formed therein. The bending portion 12 is bendable in the longitudinal and lateral directions. The flexible tube 13 exhibits the flexibility. An operation portion 15 is connected to the proximal end portion of the insertion portion 14.

The operation portion 15 functions as a grasping portion, having a universal cord 15a extending from the side portion. A not shown connector is attached to the proximal end of the universal cord 15a which is detachably connected to the control unit 20 through the connector.

The operation portion 15 is provided with two bending knobs 16 for bending the bending portion 12 in the longitudinal and lateral directions, respectively, a fluid control button 17 including an air/water feed button for feeding air/water and a suction button for suction, and various switches 18 for controlling the endoscopic image shot by an image pickup device (not shown) attached to the distal end portion 11. A treatment instrument insertion port 19 is provided to the insertion portion side of the operation portion 15. The treatment instrument insertion port 19 is communicated with the proximal end portion of the treatment instrument channel 14a within the insertion portion 14. The distal end portion of the treatment instrument channel 14a is communicated with the distal end opening 11a.

The treatment instrument 40 according to the embodiment may be, for example, a biopsy forceps introduced into the body cavity via the treatment instrument channel 14a. Hereinafter, the explanation will be made with respect to a biopsy forceps 40FB as the treatment instrument. The biopsy forceps 40FB includes a treatment section 41 structured to open and close a pair of biopsy cups 41a and 41b for taking the body tissue at the distal end portion of the sheath 43 formed as the flexible tube body that exhibits a predetermined elasticity. The sheath 43 of the biopsy forceps 40FB is wound within the sheath storage portion 61 of the cartridge 60. A not shown operation wire is inserted in the sheath 43. The operation wire is driven forward and backward through the operation of a handle 42.

The handle 42 is formed including a finger ring 44 and a slider 45. The finger ring 44 includes a hole 44a which accommodates the finger, for example, the thumb of the user. The finger ring 44 has a built-in RFID (Radio Frequency Identification) 49 serving as the IC chip which contains the treatment instrument ID in the form of the integrated circuit. The RFID 49 registers the treatment instrument information.

The slider 45 has a recess portion 45a which accommodates the mid finger and the medicinal finger of the user. The open/close state of the treatment section 41 is switched by movement of the operation wire accompanied with the backward/forward driving operation of the slider 45 of the handle 42.

The cartridge 60 which contains the sheath storage portion 61 for winding the sheath 43 to be stored is formed as a case formed as a hollow substantially cylindrical shape. The cartridge 60 includes two sheath insertion portions communicated with the sheath storage portion 61. One of the sheath insertion portions is formed in a block-like treatment instrument holding portion 62 extending from the side peripheral surface of the cartridge 60, and the other sheath insertion portion is formed in the sheath outlet 63 which protrudes upward from substantially center of the cartridge 60.

The controller 30 is structured to be connectable to the insertion portion 14 of the endoscope 10. The controller 30 is formed of a rigid main body 31 and a grip body 32 as an elastic member connected to the main body 31. A signal cable 21 extends from the grip body 32, having the proximal end portion provided with a connector (not shown). The signal cable 21 is detachably connected to the control unit 20 via the connector.

The side peripheral surface of the main body 31 is provided with an operation command unit 34 with an operation lever 33, and a rotary command unit 36, respectively. The operation lever 33 is of joy stick type which is operated through tilting as the switch of zero return type which allows biaxial operation. Accompanied with the tilting operation of the operation lever 33, the controller 30 outputs the command signal for moving the sheath 43 forward/backward or the command signal for operating the treatment section 41 via the signal cable 21.

The rotary command unit 36 is formed on the outer circumference of the controller opposite the operation lever 33. The rotary command unit 36 is formed of a rotary lever 36a and a lever support 36b. The rotary lever 36a is tiltably operated forward and backward around the axis orthogonal to the axis of the controller 30B. The lever support 36b rotatably supports the rotary lever 36a.

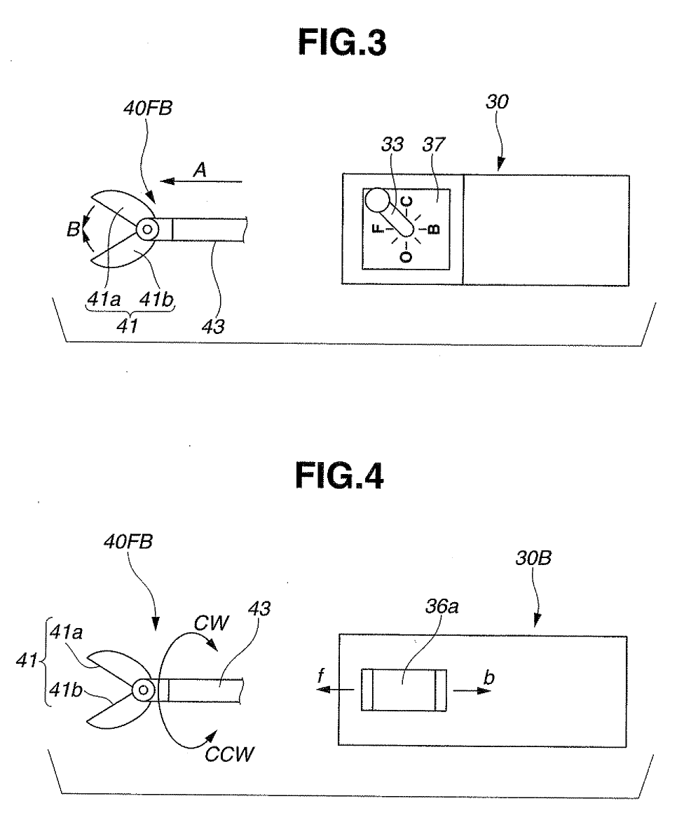

Referring to FIG. 3, an index plate 37 which indicates the tilting direction of the operation lever 33 is provided on the upper surface of the operation command unit 34 for commanding the operation of the biopsy forceps 40FB. The index “F” indicating the forward direction is marked at the distal-end side, the index “B” indicating the backward direction is marked at the rear side, the index “O” indicating the opening operation is marked at the left side with respect to the distal-end side when viewed from above, and the index “C” indicating the closing operation is marked at the right side with respect to the distal-end side, respectively.

The operator is allowed to move the sheath 43 forward and backward with respect to the insertion portion 14 of the endoscope 10 by tilting the operation lever 33 in the index “F” direction (hereinafter referred to as forward), or in the index “B” direction close to the operator (hereinafter referred to as backward). In other words, the operator is allowed to lead out the treatment section 41 from the distal end opening 11a. Meanwhile, the operator is allowed to close or open the treatment section 41 by tilting the operation lever 33 in the index C direction (hereinafter referred to as rightward) or in the index O direction (leftward).

When the operation lever 33 is tilted to the direction between the index F and the index C as shown in the drawing, the controller 30 outputs the command signal for moving the sheath 43 forward and the command signal for closing the treatment section 41 to the control unit 20. As a result, the treatment section 41 of the biopsy forceps 40FB in the opened state is moved forward as arrow A shows, and the opened state is switched to the closed state as arrow B shows.

The indexes on the index plate 37 may be changed into various forms depending on the type of the treatment instrument 40 to be used. The controller 30 in the embodiment is of the wired type for outputting the command signal to the control unit 20 via the signal cable 21. However, the controller 30 may be of wireless type rather than the wired type.

Meanwhile, when the rotary lever 36a is operated toward the direction of arrow f as the forward direction as shown in FIG. 4, the treatment section 41 is tilted clockwise as viewed from the operator. When the rotary lever 36a is tilted toward the direction of arrow b, the treatment section 41 is turned counterclockwise as viewed from the operator. The electric driver 70 is provided integrally with the sheath outlet portion 63 of the cartridge 60, for example. An electric cable 24 which contains the signal line extends from the electric driver 70. The proximal end portion of the electric cable 24 is detachable with respect to the control unit 20 via the connector (not shown). The electric driver 70 is provided with a pair of rotatable rollers 71a, 71b inside the case. The rollers 71a and 71b are formed of elastic resin members.

The electric driver 70 is provided with a not shown motor as a drive source for rotating one of the pair of rollers 71, for example, the roller 71a. The motor is driven based on the control signal output from the control section 20a of the control unit 20 to the motor in response to the command signal output to the control unit 20 accompanied with the tilting operation of the operation lever 33 of the controller 30 in the longitudinal direction. The sheath 43 of the biopsy forceps 40FB, thus, is moved forward or backward.

The electric driver 70 is provided with a rotary detection sensor 72 for detecting a rotation speed of the roller 71a, for example. The detection value of the rotary detection sensor 72 is outputted to the control unit 20 via not shown signal line inserted into the electric cable 24. Based on the detection value from the rotary detection sensor 72, the control section 20a of the control unit 20 calculates the displacement of the sheath 43 of the biopsy forceps 40FB for controlling the displacement of the sheath 43.

The sheath 43 of the biopsy forceps 40FB lead out from the sheath outlet 63 of the cartridge 60 is introduced into the case of the electric driver 70 to be interposed between the pair of rollers 71a and 71b which grip the sheath 43 therebetween under pressure. When the motor in the electric driver 70 is rotated in the desired direction in the gripped state, the sheath 43 gripped between the rollers 71a and 71b is moved forward or backward accompanied with the rotation of the roller 71a. The sheath 43 moved forward is introduced into the communication hole of a convex portion 73.

The rotary sensor 72 for detecting the rotation speed of the roller 71 is employed as the sensor for detecting the displacement of the sheath 43. However, the sensor for detecting the displacement is not limited to the rotary detection sensor 72. For example, the sensor for counting the equally spaced indexes on the surface of the sheath 43, or the sensor for detecting fine concavity and convexity formed on the surface of the sheath 43 may be employed. The motor may be provided with the encoder so as to detect the displacement of the sheath 43 by directly detecting the rotation speed of the motor shaft.

The electric opening/closing rotary device 50 having the handle 42 of the biopsy forceps 40FB set is electrically coupled with the control unit 20 via the communication cable 22 and the electric cable 23. More specifically, one end of the communication cable 22 is connected to a ring presser portion 52, and one end of the electric cable 23 is connected to a holding box 53.

The electric opening/closing rotary device 50 is formed of a hat-shaped base body 51, the ring presser portion 52, the holding box 53, a rack 54, a slider presser portion 55, a not shown drive motor, and a rotary motor 59. The ring presser portion 52, clamp members 56a, 56b, and a mount portion 57 are integrally provided on one surface of the base body 51. The rotary motor 59 is integrally provided on the other surface of the base body 51.

The ring presser portion 52 is provided with the finger ring 44 of the treatment instrument 40. The outer diameter of the ring presser portion 52 is set to be substantially the same as the inner diameter of the hole 44a of the finger ring 44 so as to firmly hold the handle 42 of the treatment instrument 40. The ring presser portion 52 is provided with a treatment instrument ID sensor (hereinafter referred to as a reading sensor) 52a as an RFID reader for reading the treatment instrument information registered in the RFID 49 built in the finger ring 44. When the finger ring 44 is brought into close to the ring presser portion 52, the reading sensor 52a reads the information registered in the RFID 49 in the non-contact manner, and outputs the read information to the control unit 20.

In the state where the handle 42 of the treatment instrument 40 is fixed to the electric opening/closing rotary device 50, the distal end side of the handle 42 is disposed on the mount portion 57. In the aforementioned state, the handle 42 of the biopsy forceps 40FB is apart from the base body 51 by a predetermined distance, and substantially in parallel with one surface of the base body 51.

The outer diameter of the ring presser portion 52 may be slightly smaller than the inner diameter of the finger ring 44 such that the elastic tube is used to cover the outer periphery of the ring presser portion 52 to hold the handle 42 of the treatment instrument 40 with the elastic force.

The slider presser potion 55 is linked to the proximal end side of the rack 54 with a setscrew 54b, for example. The slider presser portion 55 grips the slider 45 of the treatment instrument 40. Specifically, the slider presser portion 55 is provided with a pair of holding plates 55a extending to the base body 51 for gripping the recess portion 45a of the slider 45.

The holding box 53 is fixed to the base body 51 via the clamp members 56a, 56b integrally fixed thereto. The holding box 53 allows the rack 54 to move straight. The holding box 53 is provided with a not shown drive motor for driving the rack 54 held to move straight forward or backward. The motor shaft of the drive motor is provided with a pinion gear 58 in mesh with the straight gear of the rack 54. The pinion gear 58 is stored in the holding box 53.

The drive motor is driven based on the control signal outputted from the control unit 20 to the drive motor in response to the command signal outputted to the control unit 20 accompanied with the tilting operation of the operation lever 33 of the controller 30. More specifically, when the drive motor is driven by the tilting operation of the operation lever 33 in the state where the pinion gear 58 is in mesh with the straight gear 54a, the rack 54 starts moving with respect to the holding box 53 accompanied with the rotation of the pinion gear 58. As the rack 54 moves, the slider presser portion 55 moves forward or backward to move the slider 45 of the treatment instrument 40 held by the slider presser portion 55 along the shaft of the handle 42. This makes it possible to open and close the biopsy cups 41a and 41b which form the treatment section 41 of the biopsy forceps 40FB.

The rotary motor 59 rotates the sheath 43 and the treatment section 41 from the distal end portion of the handle 42 of the treatment instrument 40 around the long axis of the sheath 43. The rotary motor 59 is electrically coupled with the control unit 20 via the electric cable 23a substantially integrally provided with the electric cable 23. A rotary transfer gear 59a as a spur gear is fixed to the end of the motor shaft of the rotary motor 59.

The base body 51 has a hole 51a for exposing the rotary transfer gear 59a so as to be in mesh with a driven gear 42a. The driven gear 42a is provided at the distal end portion of the handle 42 of the treatment instrument 40. The base body 51 is provided with a holding portion 56c for rotatably holding the distal end portion of the handle 42 of the treatment instrument 40.

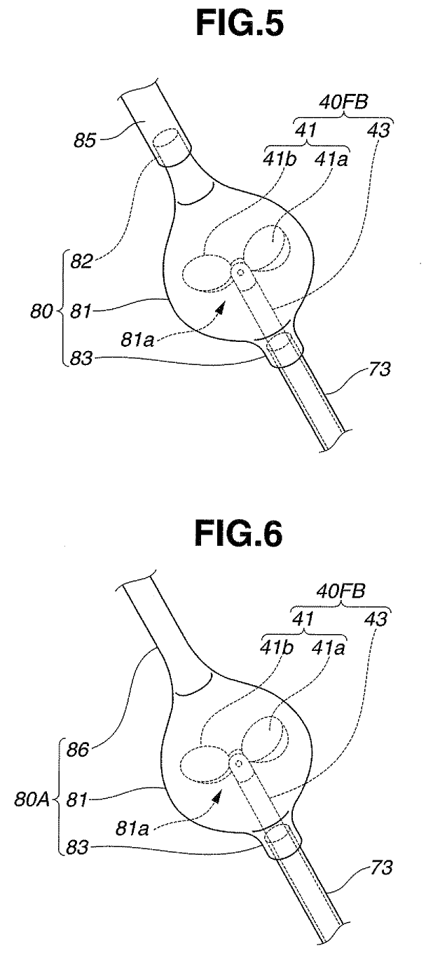

The treatment section operation check instrument 80 including the chamber 81 is formed of a transparent rigid resin member, for example, ABS resin, polycarbonate resin and the like. Referring to FIGS. 2 and 5, the treatment section operation check instrument 80 includes two communication sections communicated with a hollow section 81a of the chamber 81.

One of the communication sections is formed in an outlet 82 with which the proximal end portion of the connecting tube 85 as the connecting member is externally fit. The other communication section is formed in an inlet 83 with which the convex portion 73 is internally fit. The longitudinal axis of the outlet 82 is coaxial with the longitudinal axis of the inlet 83. In the embodiment, the hollow section 81a of the chamber 81 serves as the treatment section operation check space for confirming whether or not the biopsy cup as the treatment section 41 of the biopsy forceps 40FB is opened/closed. That is, the volume of the hollow section 81a is set to allow the treatment section 41 to perform the biopsy operation.

In the embodiment, the chamber 81 has substantially a spherical shape as shown in FIG. 2, and the inlet 83 of the chamber 81 has the semi-spherical shape. Meanwhile, at least the inner surface of the outlet 82 is formed to substantially conical shape having the diameter gradually decreased toward the outlet 82.

In the aforementioned treatment section operation check instrument 80, the convex portion 73 is internally fit with the inlet 83. Then the treatment section 41 and the sheath 43 led out from the convex portion 73 accompanied with the rotation of the motor is smoothly guided into the hollow section 81a via the inlet 83. Meanwhile, the inner surface of the outlet 82 side of the chamber 81 is substantially conical shaped, and the connecting tube 85 is externally fit with the outlet 82. Accordingly the treatment section 41 and the sheath 43 guided into the hollow section 81a may be smoothly movable from the inside the chamber 81 to the connecting tube 85 accompanied with the rotation of the motor.

The distal end portion of the connecting tube 85 is attached to the treatment instrument insertion port 19 of the endoscope 10 as shown in FIGS. 1 and 2. The treatment section 41 and the sheath 43 which move forward in the connecting tube 85 are introduced into the treatment instrument channel 14a.

The control unit 20 including the control section 20a is further equipped with a light source unit and a video processor which are not shown. The control unit 20 is connected to a display unit such as a liquid crystal display (not shown) for displaying the endoscopic image. The control section 20a is formed including a memory section, an arithmetic processing section, a determination section, and a signal input section and the like.

The function of the above-structured endoscopic system 1 will be described hereinafter.

Firstly, the staff prepares the endoscope 10 for the inspection, the controller 30, the cartridge 60 which stores the treatment section 41 of the biopsy forceps 40FB and, the sheath 43, the electric opening/closing rotary device 50, the electric driver 70, the treatment section operation check instrument 80, and the connecting tube 85 and so on.

Then the staff connects the universal cord 15a of the endoscope 10 to the control unit 20, and installs the electric driver 70 integrally with the sheath outlet 63 of the cartridge 60. The sheath 43 stored in the sheath storage portion 61 of the cartridge 60 is gripped between the rollers 71a and 71b of the cartridge 60. This makes it possible to place the treatment section 41 in the convex portion 73.

Subsequently, the staff sets the handle 42 of the biopsy forceps 40FB attached to the end of the sheath 43 extending from the treatment instrument holding portion 62 of the cartridge 60 in the electric opening/closing rotary device 50 in a predetermined state. The outlet 83 of the treatment section operation check instrument 80 is attached to the convex portion 73. The staff further communicates the distal end portion of the connecting tube 85 with the treatment instrument insertion port 19 of the endoscope 10 while communicating the proximal end portion with the outlet 82 of the treatment section operation check instrument 80. The controller 30 is connected to the insertion portion 14 of the endoscope 10. Thereafter, the staff connects the signal cable 21, the communication cable 22, and the electric cables 23, 24 to the control unit 20. Then the endoscopic system 1 as shown in FIGS. 1 and 2 is structured.

For the purpose of conducting the inspection with the endoscopic system 1, the operator turns the control unit 20 ON by operating the foot switch (not shown), for example. Then the operator tilts the operation lever 33 of the controller 30 forward to determine whether or not the biopsy cups which form the treatment section 41 is opened/closed such that the treatment section 41 of the biopsy forceps 40FB in the convex portion 73 is moved forward. The operator then sets the treatment section 41 to the desired position in the hollow section 81a by appropriately operating the operation lever 33.

The operator tilts the operation lever 33 in the lateral direction while keeping the treatment section 41 set in the hollow section 81a. Then the biopsy cups 41a and 41b of the treatment section 41 are opened/closed accompanied with the tilting operation of the operation lever 33. This makes it possible to confirm with respect to the operation of the treatment section 41.

Next, the operator opens the biopsy cups 41a and 41b as shown in FIG. 5 by tilting the operation lever 33 leftward, and further performs the tilting operation of the rotary lever 36a of the controller 30 forward and backward repeatedly. Then the treatment section 41 rotates clockwise or counterclockwise accompanied with the tilting operation of the rotary lever 36a. The operator is allowed to confirm the rotary operation of the treatment section 41.

Thereafter, the operator tilts the operation lever 33 of the controller 30 rightward to close the treatment section 41, and tilts the operation lever 33 forward again to insert the treatment section 41 and the sheath 43 into the treatment instrument channel 14a via the connecting tube 85.

Subsequently, the operator inserts the insertion portion 14 of the endoscope 10 into the body cavity of the subject body toward the target site while observing the endoscopic image. The operator brings the distal end portion of the insertion portion 14 to face the tissue of the target site, and then collects the tissue by properly performing the tilting operation of the operation lever 33 while observing the endoscopic image.

In the case where opening/closing rotary operation of the biopsy cups 41a and 41b which form the treatment section 41 cannot be confirmed in the state where the treatment section 41 is set in the hollow section 81a, the operator instructs the staff to confirm whether or not there is an error in the electric coupling between the electric cables 23, 23a and the control unit 20, or whether or not the handle 42 is correctly set in the electric opening/closing rotary device 50 and the like. In case of necessity, replacement of the electric cables 23, 23a is performed to restructure the endoscopic system 1 available for the inspection.

In the endoscopic system where the treatment section of the treatment instrument and the sheath stored in the storage portion of the cartridge is moved forward and backward by the electric driver to allow the treatment instrument electric opening/closing rotary device to perform the opening/closing operation or the rotary operation of the treatment section, the treatment section operation check instrument with the hollow section between the treatment instrument insertion port of the endoscope and the convex portion of the electric driver is provided. In the aforementioned structure, the treatment section of the treatment instrument stored in the cartridge is disposed in the hollow section to confirm the opening/closing rotary operation of the treatment section. This makes it possible to dispose the treatment section which has been subjected to the check with respect to the opening/closing rotary operation in the treatment instrument channel. Accordingly, the operation error caused by the treatment section failing to open/close, or to rotate during the inspection may be prevented.

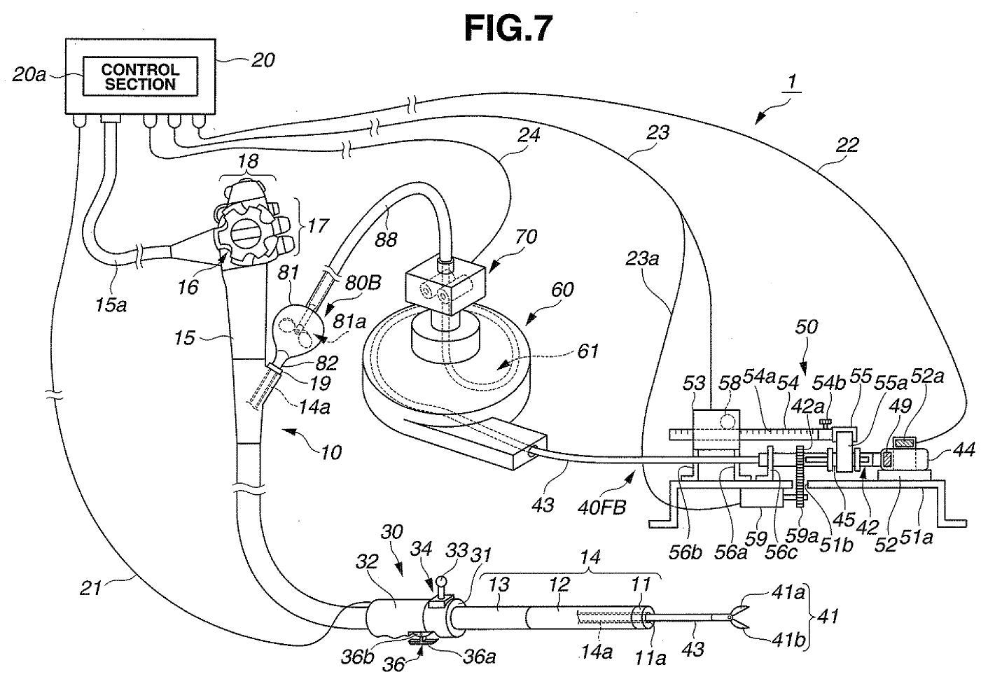

The treatment section operation check instrument 80 includes the outlet 82 and the inlet 83 such that the proximal end portion of the connecting tube 85 is inserted through the outlet 82, and the inlet 83 is attached to the convex portion 73. However, the structure of the treatment section operation check instrument 80 is not limited to the one as described above. For example, a treatment section operation check instrument 80A may be employed, having a flexible tube portion 86 formed by integrally combining the outlet 82 and the connecting tube 85 extending from the chamber 81 as shown in FIG. 6. Alternatively, a treatment section operation check instrument 80B may also be employed, having a flexible tube portion 88 formed by integrally combining the inlet 83 and the connecting tube 85 extending from the chamber 81 as shown in FIG. 7. The treatment section operation check instrument 80B allows the outlet 82 to be directly connected to the treatment instrument insertion port 19 as shown in FIG. 7.

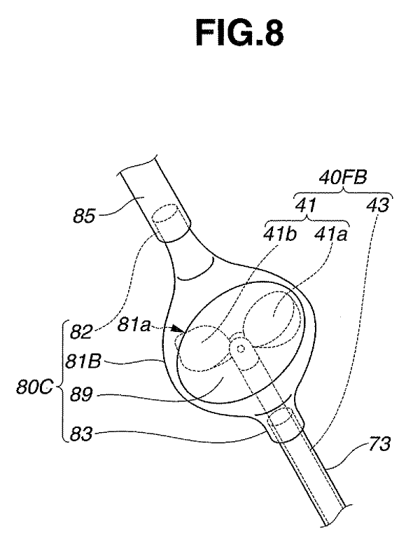

A treatment section operation check instrument 80C may be employed having a chamber 81B provided with a single lens or a pair of opposite lens portion 89 as shown in FIG. 8 for the purpose of improving the visibility of the treatment section 41 in the chamber 81. The above structure allows the operator to observe the magnified image of the treatment section 41 in the hollow section 81a through the lens portion 89. The operator is allowed to easily check the operation of the treatment section 41.

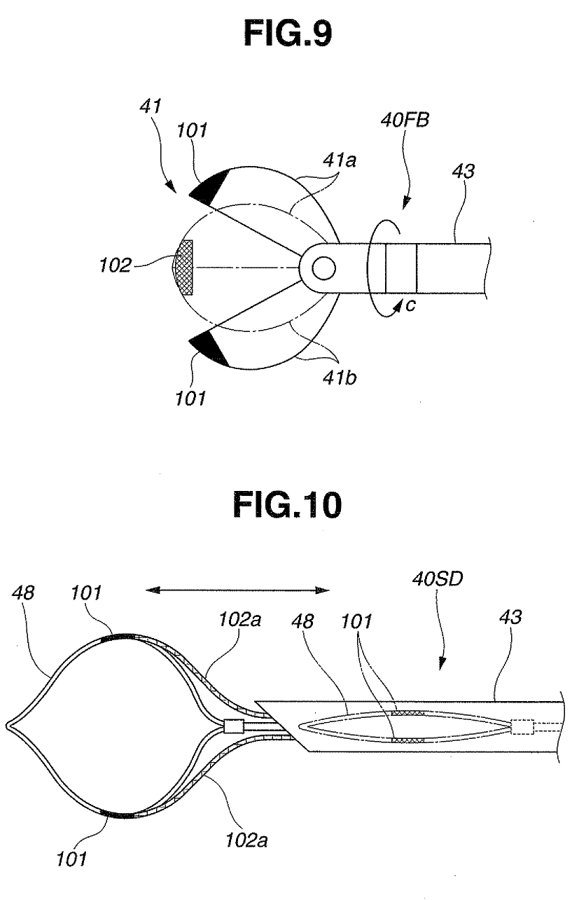

Referring to FIG. 9, a marking 101 may be formed on the treatment section 41 such that the operator is capable of easily checking the operation of the treatment section 41.

Referring to FIG. 9, the markings 101 are applied to the outer surfaces of the biopsy cups 41a and 41b which form the treatment section 41 of the biopsy forceps 40FB. In the case where the operator opens and closes the biopsy cups 41a and 41b repeatedly as indicated by the solid line and the dashed line, respectively for checking the operation, or the sheath 43 is rotated as indicated by arrow c, an after image 102 of the marking 101 is obtained. The markings 101 applied to the outer surfaces of the biopsy cups 41a and 41b allow the operator to easily check the operation by means of the afterimage 102 in the chamber 81.

In the aforementioned embodiment, the biopsy forceps 40FB is employed as the treatment instrument. However, the treatment instrument is not limited to the biopsy forceps. For example, the pair of markings 101 may be applied to a high frequency snare 40SD opposite the loop 48 in the opened state for easy determination with respect to the open state. In the aforementioned structure, when the operator moves the loop 48 forward and backward from the distal end portion of the sheath 43 as indicated by the arrow in the drawing repeatedly for checking the operation, the afterimage 102 of the marking 101 may be obtained. This makes it possible to allow the operator to easily perform the operation check as described above.

The torque wire or the multiple thread coil may be used for improving the rotating performance of the snare or the biopsy forceps.

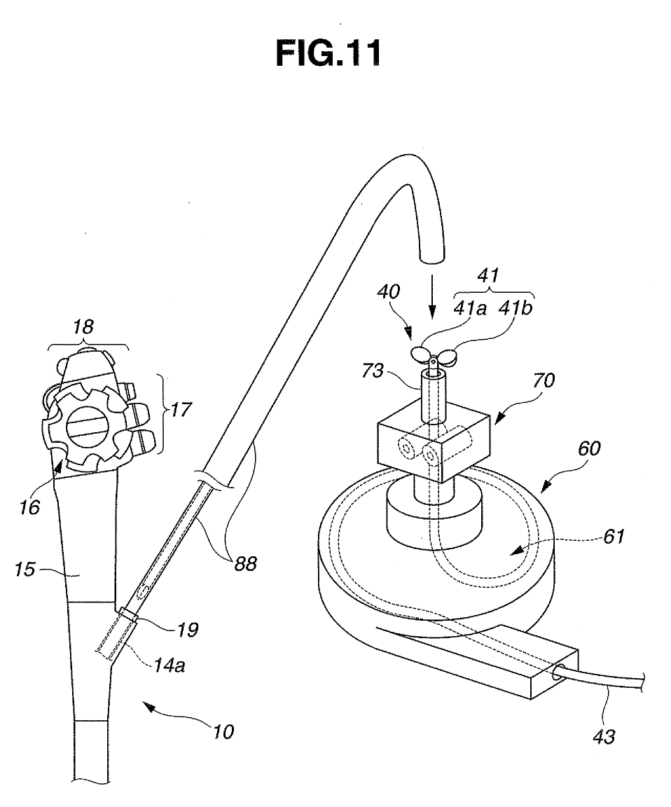

In the aforementioned embodiment, the switching rotating operation of the treatment section 41 is checked in the chamber 81 of the treatment section operation check instrument 80 interposed between the treatment instrument insertion port 19 of the endoscope 10 and the convex portion 73 of the electric driver 70. The switching rotary operation of the biopsy cups 41a and 41b may be checked by projecting the treatment section 41 from the distal opening of the convex portion 73 of the electric driver 70 as shown in FIG. 11. After checking the switching rotary operation of the treatment section 41, the treatment section 41 is disposed in the convex portion 73 again such that the proximal end portion of the connecting tube 85 is communicated with the convex portion 73. The treatment section 41 and the sheath 43 are disposed at the predetermined position in the treatment instrument channel 14a by tilting the operation lever 33.

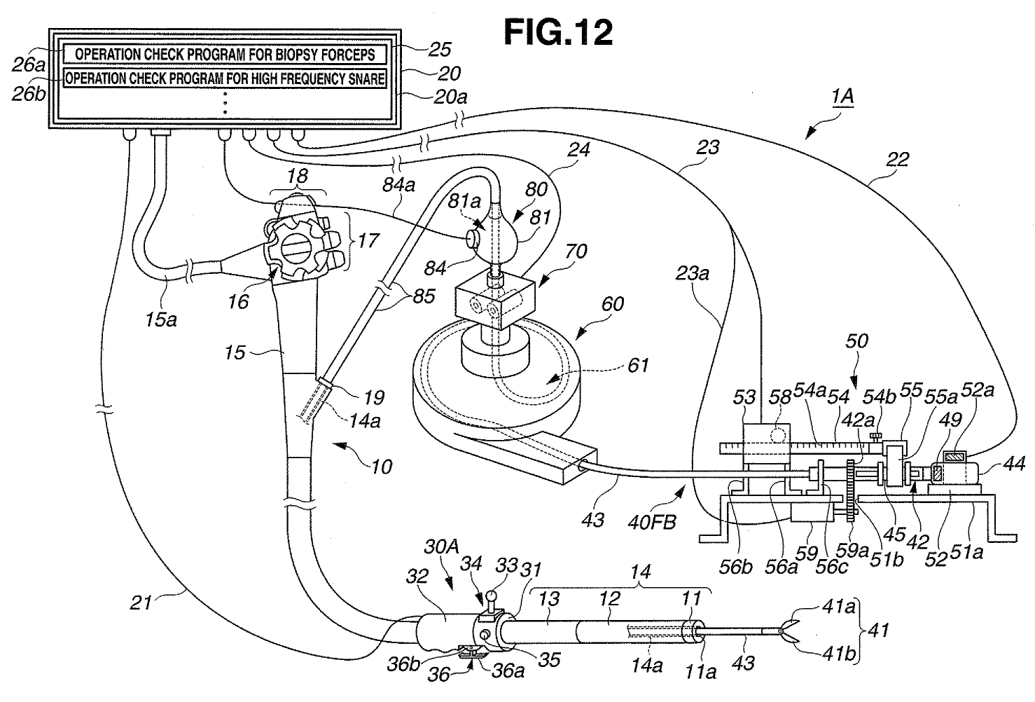

A second embodiment according to the present invention will be described referring to FIGS. 12 to 14.

In an endoscopic system 1A of the embodiment, the operation of the treatment section 41 is checked under the control of the control section 20a. A memory 25 of the control section 20a registers a treatment section operation check program 26 for performing the operation check of the treatment section in accordance with the type of the treatment instrument, for example, an operation check program 256a for the biopsy forceps, an operation check program 26b for high frequency snare and the like.

A controller 30A of the embodiment includes an operation check command button (hereinafter referred to as a check button) 35 in the form of a push switch. Once the button is pushed, a treatment section operation check start signal is outputted to the control unit 20 via the signal cable 21. That is, the switching rotary operation check of the treatment section 41 is started under the control of the control section 20a.

The chamber 81 of the treatment section operation check instrument 80 is provided with a sensor 84 as a detection portion. A signal cable 84a connected to the control unit 20 extends from the sensor 84. The sensor 84 in the form of a magnetic sensor, for example, detects whether or not the treatment section 41 has reached inside the hollow section 81a. If the treatment section 41 has reached inside the cavity 81a, the sensor 84 outputs the notification signal.

The sensor 84 is not limited to the magnetic sensor, but may be formed as an optical sensor. Other structure is the same as that of the first embodiment. The same members may be designated with the same reference numerals, and explanations thereof, thus will be omitted.

An operation of the endoscopic system 1A will be described.

Likewise the case of the endoscopic system 1, the staff prepares the endoscope 10 for the inspection, the cartridge 60 which stores the treatment section 41 which forms the biopsy forceps 40FB and, the sheath 43, the electric opening/closing rotary device 50, the electric driver 70, the treatment section operation check instrument 80, the connecting tube 85, and a controller 30A in place of the controller 30.

Then the staff performs the same operation as that for structuring the endoscopic system 1. In other words, the staff conducts the operation for connecting the universal cord 15a of the endoscope 10 to the control unit 20, the operation for integrally attaching the electric driver 70 to the sheath outlet 63 of the cartridge 60, the operation for interposing the sheath 43 stored in the sheath storage portion 61 of the cartridge 60 between the rollers 71a and 71b of the electric driver 70 to dispose the treatment section 41 in the predetermined position of the convex portion 73, the operation for setting the handle 42 of the biopsy forceps 40FB extending from the treatment instrument holding portion 62 of the cartridge 60 in the electric opening/closing rotary device 50, the operation for attaching the inlet 83 of the treatment section operation check instrument 80 to the convex portion 73, the operation for communicating the distal end portion of the connecting tube 85 with the treatment instrument insertion port 19 of the endoscope 10, the operation for communicating the proximal end portion of the connecting tube 85 with the outlet 82 of the treatment section operation check instrument 80, the operation for arranging the controller 30A to the insertion portion 14 of the endoscope 10, and the operation for connecting the signal cable 21, the communication cable 22, and the electric cables 23, 24 to the control unit 20, respectively. The endoscopic system 1A may be structured through the aforementioned operations.

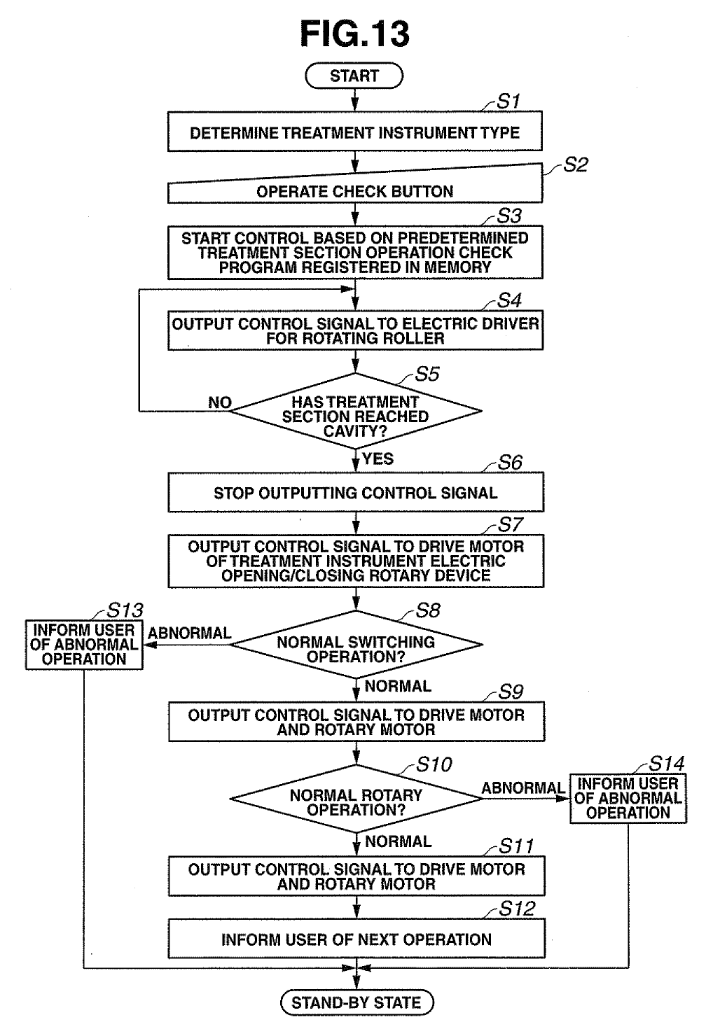

When the inspection is conducted with the endoscopic system 1A, the operator turns the control unit 20 ON by operating the not shown foot switch to start the control of the control section 20a. When the control unit 20 is turned ON, the control unit 20 receives an input of the information registered in the RFID 49 built in the finger ring 44 which has been read by the reading sensor 52a. That is, the control section 20a determines that the treatment instrument set in the electric opening/closing rotary device 50 is the biopsy forceps 40FB as shown in step S1 of FIG. 13.

Then in step S2, the operator pushes the check button 35 disposed on the controller 30A such that the treatment section operation check start signal is outputted to the control unit 20. Upon reception of the treatment section operation check start signal, the control section 20a starts the control based on the operation check program 26a for biopsy forceps corresponding to the preliminarily determined biopsy forceps 40FB selected from the treatment section operation check program 26 registered in the memory 25.

Referring to step S4, the control section 20a outputs the control signal for rotating the roller 71a to the electric driver 70. Then the treatment section 41 of the biopsy forceps 40FB disposed in the convex portion 73 is moved toward the inside of the chamber 81 of the treatment section operation check instrument 80. During the rotating operation of the roller 71a, the process proceeds to step S5 where the control section 20a monitors whether or not the notification signal outputted from the sensor 84 has been inputted. That is, the control section 20a monitors whether or not the treatment section 41 has reached inside the chamber 81.

In step S5, when the control section 20a determines that the treatment section 41 has reached inside the chamber 81 based on the signal outputted from the sensor 84, the process proceeds to step S6. In step S6, the control section 20a stops outputting the control signal for rotating the roller 71a to the electric driver 70. The process then proceeds to step S7.

In step S7, the control section 20a outputs the control signal for rotating the drive motor of the electric opening/closing rotary device 50 such that the biopsy cups which form the treatment section 41 are opened and closed repeatedly. After outputting the control signal to the drive motor in step S7, the process proceeds to step S8 where the control section 20a performs the switching operation check whether or not the opening/closing operation is in the normal state based on the current value inputted to the control unit 20 from the torque meter provided for the drive motor as the power detection means.

In step S8, if the control section 20a determines that the switching operation of the biopsy cups is in the normal state, the process proceeds to step S9. In step S9, the control section 20a outputs the control signal to the drive motor for bringing the biopsy cups into the opened state, and outputs the control signal to the rotary motor 59 for rotating the biopsy cups in the opened state clockwise and counterclockwise repeatedly. This makes it possible to rotate the sheath 43 and the treatment section 41 clockwise and counterclockwise repeatedly for a predetermined period.

The control unit 20a outputs the control signal to the drive motor and the rotary motor 59 in step S9. The process then proceeds to step S10 where the current value inputted to the control unit 20 from the torque meter of the rotary motor 59 is measured, and the rotary operation check is made whether or not the rotary operation is in the normal state.

In step S10, if the control section 20a determines that the rotating operation of the biopsy cups is in the normal state, the process proceeds to step S11. In step S11, the control section 20a outputs the control signal to the drive motor for closing the biopsy cups to stop driving, and outputs the control signal to the rotary motor 59 for stopping repetitive clockwise and counterclockwise rotations. As a result, the biopsy cups may be disposed in the chamber 81 without being rotated in the closed state.

After performing the notification processing to notify the operator of the subsequent operation as shown in step S12, the control section 20a is brought into the stand-by state to end the treatment section operation check program. In the stand-by state, the control section 20a waits for the command signal from the controller 30.

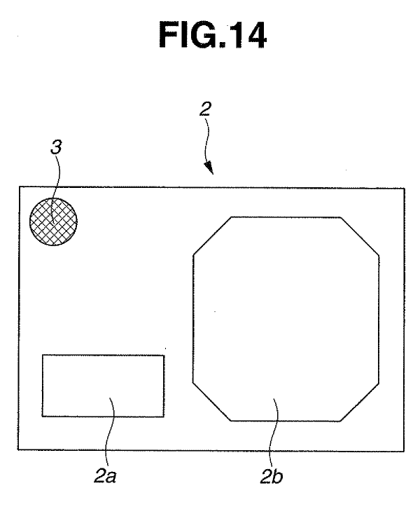

The notification process in step S12 is performed for notifying the operator and the staff of the subsequent inspection. In the process, such message as “Ready for starting inspection” may be displayed on a subscreen 2a of the display unit 2 as shown in FIG. 14, or a certain melody for prompting the inspection may be sounded from the buzzer 3 formed on the display unit 2. The notification process allows the operator and the staff to confirm that the treatment section operation check program has ended. The reference numeral 2b denotes the main screen for observing the endoscopic image.

After confirming the end of the treatment section operation check program, the operator inserts the insertion portion 14 of the endoscope 10 toward the target site in the body cavity of the subject while observing the endoscopic image. After bringing the distal end portion of the insertion portion 14 to face the tissue of the target site, the operator collects the tissue by appropriately tilting the operation lever 33 while observing the endoscopic image. Meanwhile, after the switching operation check in step S8, or the rotary operation check in step S10, if the control section 20a detects the abnormality in the switching operation or the rotary operation of the treatment section 41, the process proceeds to steps S13 and S14 where the control section 20a performs the abnormality notification process, and is brought into the stand-by state.

The abnormality notification process in steps S13 and S14 is intended to notify the operator and the staff of the abnormality in the operation of the treatment section. For example, such messages as “Check the treatment section”, “Check the switching operation” or “Check the rotary operation” may be displayed on the subscreen 2a of the display unit 2 as shown in FIG. 14, or the alarm may be intermittently sounded from the buzzer 3 so as to inform occurrence of the abnormality.

As described above, the treatment section and the sheath of the treatment instrument stored in the storage portion of the cartridge is checked by operating the electric driver and the treatment section electric opening/closing rotary device based on the control signal outputted from the control unit according to the treatment section operation check program corresponding to the treatment instrument registered in the memory of the control unit. This makes it possible to dispose the treatment section in the treatment instrument channel of the endoscope after confirming the operation of the treatment section by the control unit. Accordingly, the error caused by the state where the treatment section is not operated during the inspection may be prevented.

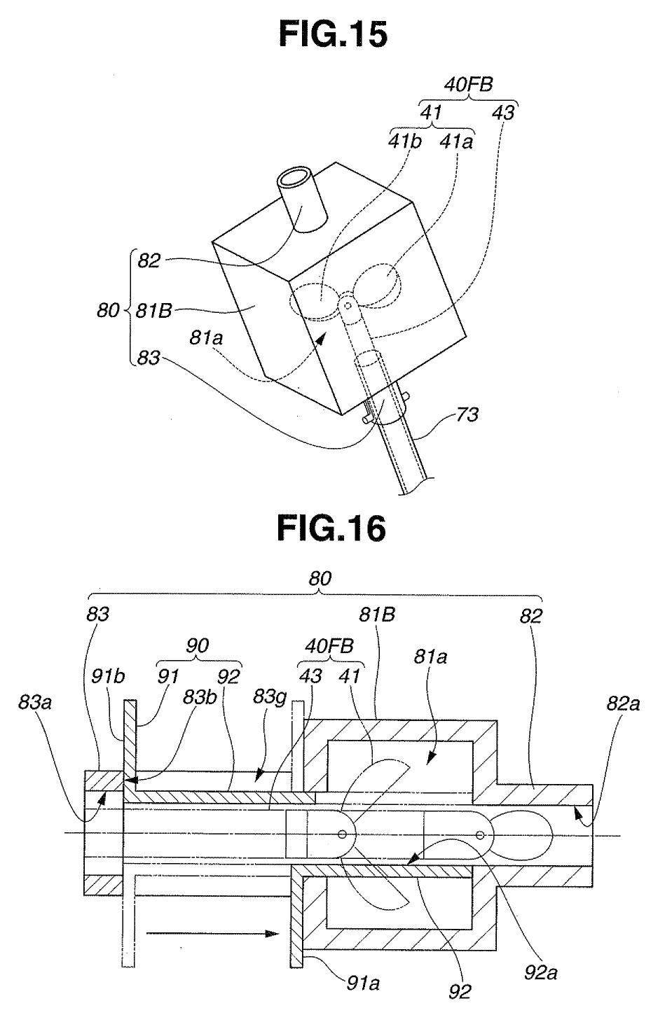

In the present embodiment, the chamber 81 has substantially a spherical shape. However, the chamber 81 is not limited to the substantially spherical shape but may be formed to have the cubic shape as a chamber 81B shown in FIG. 15.

In the aforementioned structure, preferably a retractable shutter member 90 is disposed in the hollow section 81a of the chamber 81B as shown in FIG. 16. The shutter member 90 is formed including a knob 91 and a cylindrical passage-forming portion 92. Referring to FIGS. 15 and 16, the knob 91 is formed of a pair of rod-shaped protrusions each protruding from the outer peripheral surface of the passage-forming portion 92, which face each other sandwiching the longitudinal axis of the shutter member 90. The passage-forming portion 92 is slidably arranged in an inner bore 83a of the inlet 83, for example. The knob 91 protrudes from a notched groove 83g having an open end surface formed in the outer peripheral surface of the inlet 83 so as to be sidably arranged.

The shutter member 90 is arranged such that a proximal end surface 91b of the knob 91 abuts on a bottom surface 83b of the notched groove 83g as shown in the upper half portion of FIG. 16. That is, in the state where the shutter member 90 is not protruded into the hollow section 81a, the switching rotary operation check is performed by keeping the treatment section 41 in the opened state as shown by the chain double-dashed line in the hollow section 81a. The switching rotary operation check is performed based on the measured current value inputted from the torque meter to the control unit 20.

Upon the end of the operation check of the treatment section 41, the operator closes the treatment section 41 to move the shutter member 90 toward the hollow section 81a as shown by the arrow in the lower half of FIG. 16 while grasping the knob 91. That is, the sheath 43 and the treatment section 41 are disposed in an inner bore 92a of the passage-forming portion 92 by protruding the shutter member 90 into the hollow section 81a. Thereafter, a distal end surface 91a of the knob 91 abuts on the chamber 81. Then referring to the solid line, the passage-forming portion 92 is disposed in the hollow section 81a for communicating the inner bore 83a of the inlet 83 with the inner bore 82a of the outlet 82. The treatment section 41 and the sheath 43 which have been subjected to the operation check may be guided into the inner bore 82a of the outlet 82 as indicated by the solid line shown in the drawing.

As the movable shutter member is disposed in the chamber, the operation check of the treatment section in the hollow section may be performed, and the treatment section and the sheath may be smoothly guided outside the hollow section after the operation check.

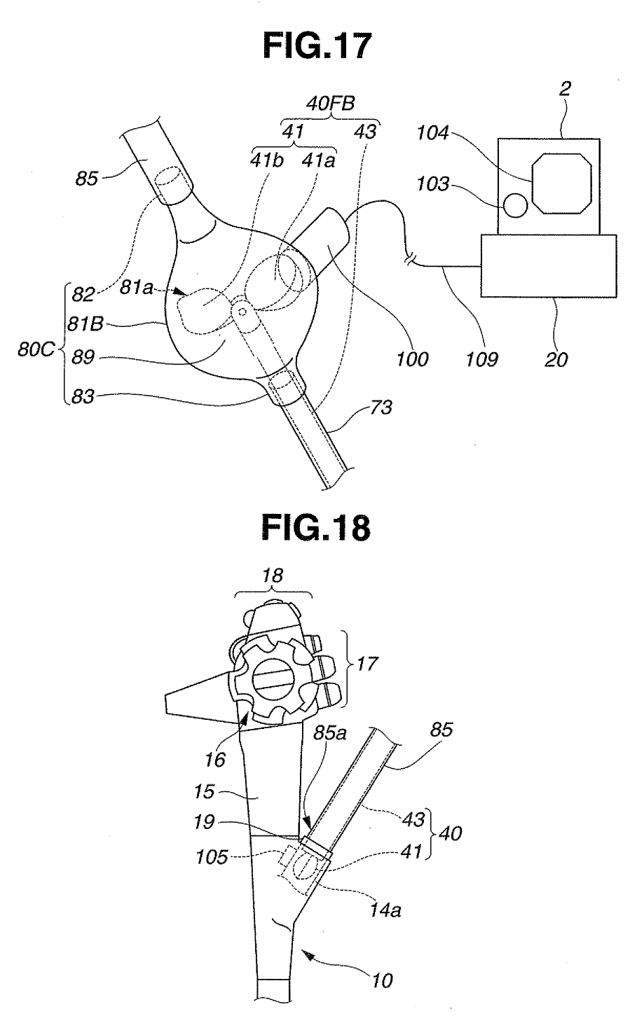

In the aforementioned embodiment, the sensor 84 or the like is disposed on the chamber 81 for detecting whether or not the treatment section 41 has reached inside the hollow section 81a. The determination whether or not the treatment section 41 has reached inside the hollow section 81a may not be limited to be made by the sensor 84 but may be made by an observation camera 100 as the detector for observing the inside of the hollow section 81a in the chamber 81 as shown in FIG. 17. A camera cable 109 extending from the observation camera 100 is connected to the control unit 20.

The observation camera 100 attached to the chamber 81 is structured to shoot the image of the treatment section so as to detect whether or not the treatment section 41 has reached inside the hollow section 81a. The determination whether or not the treatment section 41 in the hollow section 81a is operated as specified is made by observing the image of the treatment section shot by the observation camera 100. The image inside the hollow section 81a shot by the observation camera 100 is displayed on a display 103 for the image shot by the camera provided for the display unit 2. A reference numeral 104 denotes the screen for displaying the endoscopic image.

In the aforementioned embodiment, the treatment section 41 is protruded from the distal end opening of the convex portion 73 of the electric driver 70 so as to perform the operation check of the biopsy cups 41a and 41b as shown in FIG. 11. A sensor 105 may be provided in the vicinity of the treatment instrument insertion port 19 of the treatment instrument channel 14a so as to detect that the treatment section 41 has been adjacent thereto as shown in FIG. 18. The sensor 105 may be a magnetic sensor structured to output the detection signal to the control unit 20 when the treatment section 41 is brought to be adjacent thereto.

This makes it possible to perform the operation check of the treatment section in the following procedures.

Firstly, the operator moves the treatment section 41 and the sheath 43 disposed in the convex portion 73 forward by tilting the operation lever 33. The treatment section 41 which is moved forward passes through the connecting tube 85 and the treatment instrument insertion port 19 so as to be inserted into the treatment instrument channel 14a. The sensor 105 disposed in the vicinity of the treatment instrument insertion port 19 outputs the detection signal to the control unit 20. Under the control of the control section 20a, the forward movement of the treatment section 41 and the sheath 43 is stopped, and the buzzer is sounded. The buzzer allows the operator to determine that the treatment section 41 is disposed in the vicinity of the treatment instrument insertion port 19 as shown in FIG. 18.

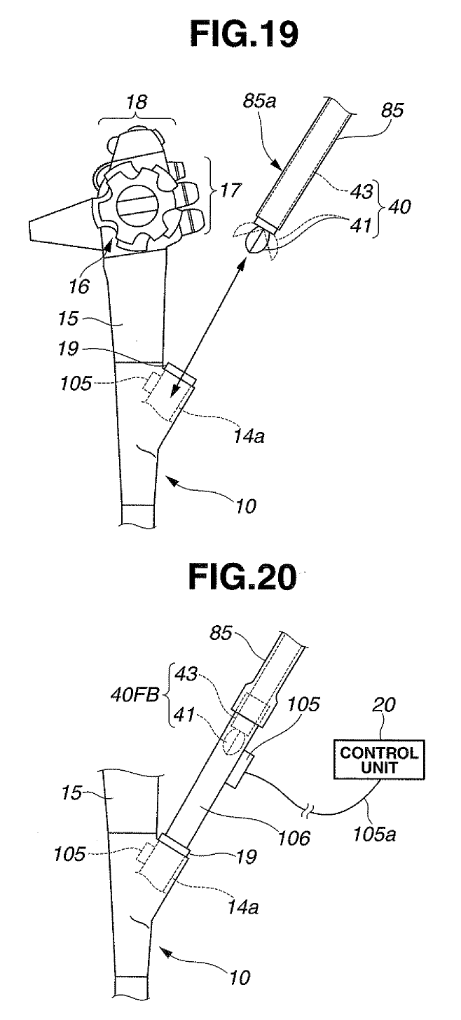

Next, the operator detaches the distal end portion 85a of the connecting tube 85 from the treatment instrument insertion port 19. Then distal end portions of the treatment section 41 and the sheath 43 are protruded from the distal end portion of the connecting tube 85 as shown in FIG. 19.

Subsequently, after confirming that the distal end portions of the treatment section 41 and the sheath 43 are protruded, the operator checks the switching rotary operation of the treatment section 41 by operating the operation lever 33 and the rotary lever 36a. When the switching rotary operation check is completed, the operator inserts the treatment section 41 into the treatment instrument channel 14a again, and attaches the distal end portion 85a of the connecting tube 85 to the treatment instrument insertion port 19 again. The operator then arranges the treatment section 41 and the sheath 43 at the predetermined position in the treatment instrument channel 14a by operating the operation lever 33.

Thus, the sensor disposed in the vicinity of the treatment instrument insertion portion allows the operation check of the treatment section protruded from the connecting tube detached from the endoscope.

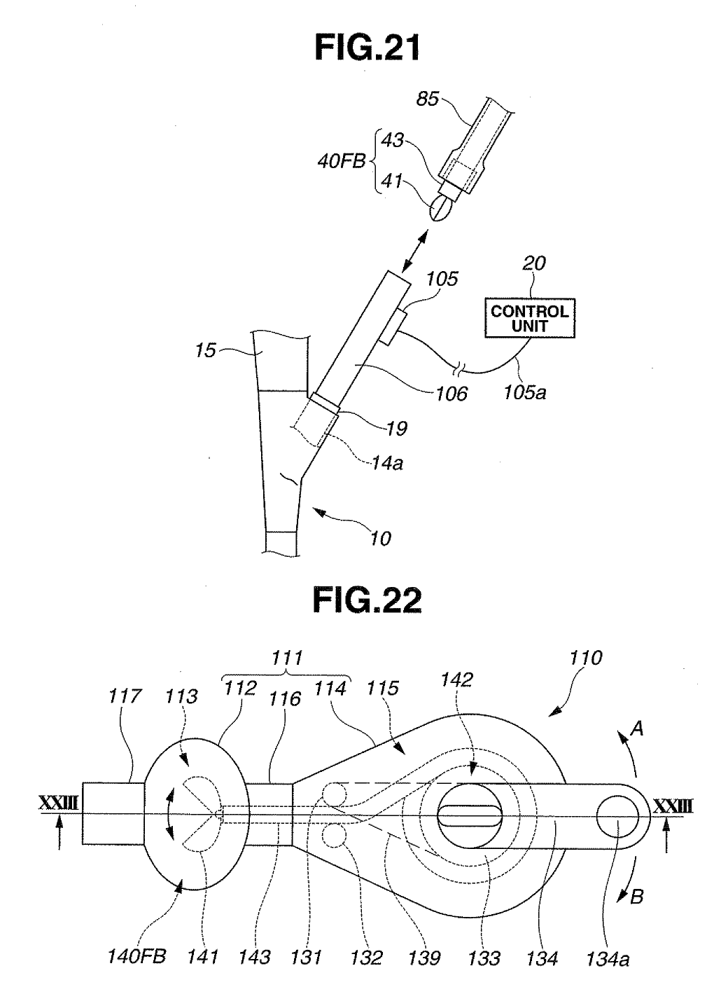

In the aforementioned embodiment, the sensor 105 is disposed in the vicinity of the treatment instrument insertion port 19. However, the position for disposing the sensor 105 is not limited to the one in the vicinity of the treatment instrument insertion port 19. For example, the sensor 105 may be disposed at a connecting adaptor 106 detachably installed in the treatment instrument insertion port 19 as shown in FIG. 20. In the aforementioned structure, the switching rotary operation of the treatment section 41 may be checked by detaching the connecting tube 85 from the connecting adaptor 106 as shown in FIG. 21. This may provide the structure for checking the operation of the treatment section 41 without providing an additional sensor to the endoscope. A reference numeral 105a denotes the signal line through which the detection signal of the sensor 105 is outputted to the control unit 20.

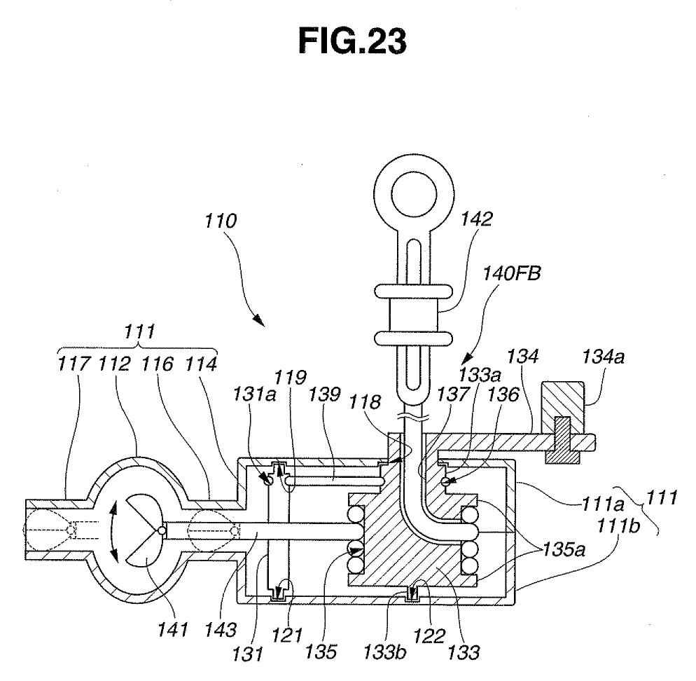

In the aforementioned embodiment, the treatment section operation check instrument 80 is provided separately from the cartridge 60. However, a cartridge with chamber as shown in FIGS. 22 and 23 may be structured where the treatment section operation check instrument 80 is formed integrally with the cartridge 60.

Referring to FIGS. 22 and 23, a structure of a cartridge with chamber 110 will be described hereinafter.

The cartridge with chamber 110 includes a main body 111, a pair of pulleys 131, 132 disposed in the main body 111, a drum 133 and a drive belt 139.

The main body 111 includes a chamber portion 112 having a hollow section 113 and a cartridge portion 114 having a sheath storage portion 115. A link portion 116 for communicating the hollow section 113 and the sheath storage portion 115 is formed between the chamber portion 112 and the cartridge portion 114. An outlet 117 is formed at the distal end side of the chamber portion 112.

The hollow section 113 of the chamber portion 112 is formed in accordance with each shape of the treatment sections 141 of the treatment instrument 140 of various types. The sheath storage portion 115 of the cartridge portion 114 is formed in accordance with the length and the radius of each sheath 143 of the treatment instrument 140 of various types.

The main body 111 is formed of two body-forming portions 111a and 111b. A first body-forming portion 111a (hereinafter referred to as a first component) 111a and a second body forming portion (hereinafter referred to as a second component) 111b are integrally fixed through adhesion or welding to form the main body 111.

A stepped through hole 118 for communicating the outer side surface with the inner side surface is formed in the first component 111a. A large diameter portion of the through hole 118 serves as a support portion for rotatably supporting a cylindrical portion 133a as one end of the drum 133. A first pulley hole 119 is formed in the inner side surface of the first component 111a which rotatably supports the shaft portion at one end side of the pulleys 131 and 132.

Meanwhile, a second pulley hole 121 which rotatably supports the shaft portion of the other end side of the pulleys 131 and 132, and a drum hole 122 for rotatably supporting the shaft portion 133b at the other end side of the drum 133 are formed in the inner side surface of the second component 111b.

The drum 133 includes a handle 134, a reel portion 135 for winding the sheath 43, and a recess portion for accommodating the drive belt (hereinafter referred to as a belt recess portion) 136. The sheath 143 is wound around the reel portion 135. The drum 133 includes a sheath insertion hole 137 through which the sheath 143 wound around the reel portion 135 is guided outside the main body 111.

The handle 134 is integrated with the drum 133, or integrally fixed to the drum 133 through threading, soldering, adhering and the like. A handle holding portion 134a is integrally fixed to the handle 134 through the threading, for example. Alternatively, the handle holding portion 134a may be rotatably attached to the handle.

The reel portion 135 includes a pair of flanges 135a as shown in FIG. 23. The belt recess portion 136 is a peripheral groove formed in the outer peripheral surface of the cylinder portion 133a between the handle 134 and the reel portion 135. The belt recess portion 136 accommodates the drive belt 139. A peripheral groove 131a which accommodates the drive belt 139 is formed in the pulley 131.

The procedure for assembling the cartridge with chamber 110 will be described hereinafter.

The operator prepares the components 111a, 111b corresponding to the treatment instrument to be stored, the pair of pulleys 131, 132, the drum 133, and the drive belt 139. The sheath 143 of the treatment instrument 140 has been preliminarily wound around the reel portion 135 of the drum 133. In other words, the treatment instrument 140 is equipped with the drum.

Then the operator disposes the drum 133 and the pulleys 131, 132 on the inner surface side of the first component 111a, and further disposes the drive belt 139 in the belt recess portion 136 and the peripheral groove 131a.

When the drum 133 is disposed in the first component 111a, the handle 134 is disposed on the outer surface side through the through hole 118, and the cylinder portion 133a of the drum 133 is disposed in the large diameter portion of the through hole 118. The drive belt 139 is disposed on the cylinder portion 133a.

Meanwhile, the pulleys 131 and 132 are disposed having the sheath 143 interposed therebetween, and each shaft portion at one ends thereof disposed in the first pulley hole 119. The drive belt 139 is disposed in the belt recess portion 136 of the drum 133 and the peripheral groove 131a of the pulley 131 upon disposing the pulley 131 in the first pulley hole 19. As indicated by the dashed line in FIG. 23, the treatment section 141 of the treatment instrument 140 is disposed in the link portion 116.

Then the operator fixes the second component 111b integrally with the first component 111a to form the cartridge with chamber 110. At this time, the operator disposes the shaft portion of the other side of the pulleys 131, 132 in the second pulley hole 121 formed in the inner side surface of the second component 111b, and the shaft portion of the other side of the drum 133 in the drum hole 122.

As described above, the pair of rotatable pulleys 131, 132 and the drum 133 rotated by operating the handle 134 are disposed in the cartridge portion 114 of the main body 111.

The drum 133 is rotated by rotating the handle 134 to the arrow A or B direction shown in FIG. 22. The pulley 131 is rotated by moving the drive belt 139 accompanied with the rotation of the drum 133. As the pulley 131 is rotated, the sheath 143 interposed between the pulleys 131 and 132 moves forward or backward.

The operation of the cartridge with chamber 110 will be described.

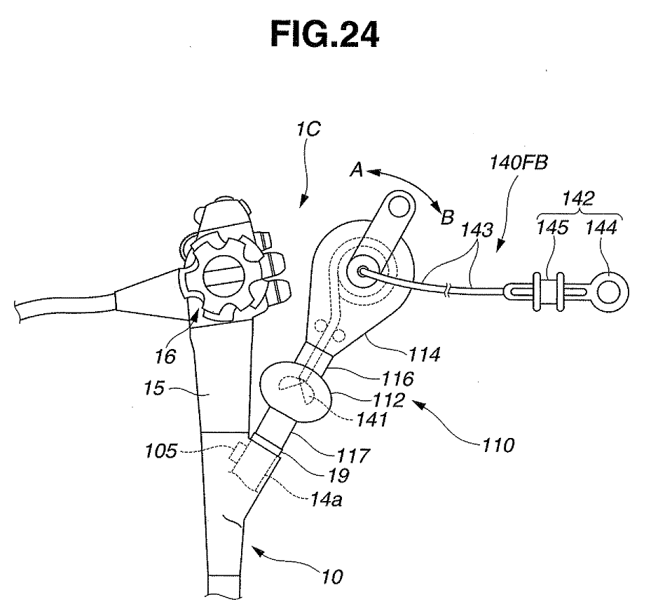

Likewise the case of the endoscopic system 1, the staff prepares the endoscope for the inspection, and the cartridge with chamber 110 which forms the biopsy forceps 140FB as the treatment instrument 140. In the embodiment, the staff structures the endoscopic system 1C by attaching the outlet 117 of the cartridge with chamber 110 to the treatment instrument insertion port 19 as shown in FIG. 24. Likewise the aforementioned embodiment, the outlet 117 may be communicated with one end of the connecting tube 85 having the other end communicated with the treatment instrument insertion port 19.

Upon inspection with the endoscopic system 1C, the operator rotates the handle 134 in the arrow A direction to confirm whether or not the biopsy cups which form the treatment section 141 are opened/closed. Accompanied with the rotation of the handle 134, the drum 133 starts rotating to move the drive belt 139, and the pulley 131 is rotated according to the movement of the drive belt 139. Then the sheath 143 interposed between the pulleys 131 and 132 is moved forward to cause the treatment section 141 of the biopsy forceps 140FB disposed in the link portion 116 to move to the inside of the hollow section 113. After confirming that the treatment section 141 has been disposed in the hollow section 113, the operator stops operating the handle 134.

Then the operator moves the handle 142 of the biopsy forceps 140FB forward or backward in the state where the treatment section 141 is set in the hollow section 113. That is, the slider 145 is moved with respect to the finger ring 144 which forms the handle 142. The forward or backward movement of the handle 142 serves to open or close the pair of biopsy cups of the treatment section 141. This allows the operator to confirm the switching operation of the treatment section 141.

Thereafter, the operator rotates the handle 142 clockwise and counterclockwise for checking the rotary operation so as to confirm whether or not the treatment section 141 rotates. Then the operator operates the handle 134 in the arrow A direction to dispose the treatment section 141 at the desired position in the treatment instrument channel 14a.

Subsequently, the operator inserts the insertion portion 14 of the endoscope 10 having the treatment section 41 of the biopsy forceps 140FB and the sheath 43 disposed in the treatment instrument channel 14a toward the target site in the body cavity of the subject while observing the endoscopic image. The operator brings the distal end portion of the insertion portion 14 to face the tissue of the target site, and properly operates the handle 142 to collect the tissue while observing the endoscopic image.

In the state where the treatment section 141 is set in the hollow section 113, when the switching rotary operation of the treatment section 141 cannot be confirmed, the operator commands the staff to replace the cartridge with chamber 110.

The cartridge with chamber is formed of the chamber portion and the cartridge portion to allow the treatment section of the treatment instrument to be disposed in the hollow section for confirming the switching operation of the treatment section. This may eliminate the work for attaching the chamber to the cartridge as described above to improve the workability. Additionally, the chamber portion optimal for the size of the treatment section may be preliminarily provided to ensure checking with no interference of the treatment section with respect to the chamber portion.

The drum and the pair of pulleys are rotatably provided in the cartridge portion such that one of the pulleys is driven to rotate accompanied with the rotation of the drum. This allows the sheath interposed between the pair of pulleys to be moved forward and backward accompanied with the rotation of the drum.

In the aforementioned embodiment, the drum is rotated by rotating the handle. However, the drum may be rotated under the driving force of the motor. The handle of the treatment section may be attached to the electric switching device or the electric opening/closing rotary device.

In the first and the second embodiments as described above, the treatment section is not disposed in the cartridge. However, the structure which allows the treatment section to be stored in the cartridge may be employed as shown in FIG. 23.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An endoscopic system comprising:
an endoscope including a treatment instrument channel which allows a treatment section and an insertion portion of a treatment instrument to be inserted;
a cartridge which stores the insertion portion of the treatment instrument; and
a treatment section operation check instrument equipped with a chamber having a hollow section with a volume which allows the treatment section to perform a treatment operation, wherein the treatment section operation check instrument equipped with the chamber is disposed between the endoscope and the cartridge for performing an operation check by disposing the treatment section in the hollow section of the chamber before inserting the treatment section into the treatment instrument channel.

2. The endoscopic system according to claim 1, wherein the treatment section operation check instrument is directly connected to the endoscope or connected to the endoscope via a connecting tube to be disposed between the endoscope and the cartridge.

3. The endoscopic system according to claim 1, wherein the treatment section operation check instrument disposed between the endoscope and the cartridge is integrated with the cartridge, directly connected to the cartridge, or connected to the cartridge via a connecting tube.

4. The endoscopic system according to claim 1, wherein the chamber of the treatment section operation check instrument includes a transparent portion which makes an inside of the hollow section visible.

5. The endoscopic system according to claim 4, further comprising:
a control unit including a control section;
a treatment instrument insertion portion electric driver for driving back and forth the treatment section of the treatment instrument under a control of the control unit;
a treatment section operation unit for allowing the treatment section of the treatment instrument to be operated under the control of the control unit; and
a treatment section operation unit for outputting a command signal to operate the treatment instrument insertion portion electric driver and the treatment section unit to the control unit.

6. The endoscopic system according to claim 4, wherein a marking is applied to the treatment section for allowing an easy operation check of the treatment section in the hollow section through the transparent portion.

7. The endoscopic system according to claim 5, further comprising a detection unit provided in the chamber of the treatment section operation check instrument for outputting a detection signal to the control unit when the treatment section of the treatment instrument is disposed in the hollow section.

8. The endoscopic system according to claim 7, wherein a marking is applied to the treatment section for allowing an easy operation check of the treatment section in the hollow section through the transparent portion.

9. The endoscopic system according to claim 7, further comprising:
a memory contained in the control section of the control unit, which registers a treatment section operation check program for performing the operation check of the treatment section in accordance with a type of the treatment instrument;
an operation check command button disposed on the treatment section operation unit for outputting a command signal to the control unit to perform the operation check by operating the treatment instrument insertion portion electric driver and the treatment section operation unit based on the treatment section operation check program registered in the memory; and
a detection unit for outputting a detection signal to the control unit when the treatment section of the treatment instrument is disposed in the hollow section, and for detecting whether or not the treatment instrument in the hollow section is operated as specified.

10. The endoscopic system according to claim 9, wherein the treatment section insertion portion electric driver is equipped with a detection unit for detecting a displacement of the treatment instrument insertion portion.

11. A treatment section operation check instrument comprising:
a chamber having a hollow section which allows a treatment section of an endoscopic treatment instrument stored in a sheath storage portion of a cartridge to be disposed, having a volume which allows the treatment section to perform a treatment operation, and includes a transparent portion which makes the treatment section in the hollow section visible; and
a pair of communication sections detachable with respect to the chamber, which are communicated with the hollow section of the chamber, wherein a longitudinal axis of one of the communication sections is coaxial with a longitudinal axis of the other communication section.

12. The treatment section operation check instrument according to claim 11, wherein the one of the communication sections communicated with the hollow section is an outlet directly connected to the endoscope or connected to the endoscope via a connecting tube.

13. The treatment section operation check instrument according to claim 12, wherein the one of the communication sections is a flexible tube formed by integrating the outlet and the connecting tube.

14. The treatment section operation check instrument according to claim 11, wherein the other one of the communication sections communicated with the hollow section is a link portion for communicating the hollow section and the sheath storage portion, or an inlet directly connected to the cartridge or connected to the cartridge via the connecting tube.

15. The treatment section operation check instrument according to claim 14, wherein the other one of the communication sections is a flexible tube formed by integrating the inlet and the connecting tube.

16. The treatment section operation check instrument according to claim 14, wherein the sheath storage portion and the hollow section are integrally provided with the link portion.

17. The treatment section operation check instrument according to claim 11, wherein the chamber includes a lens portion capable of magnifying an inside of the hollow section to be observable.

18. The treatment section operation check instrument according to claim 12, wherein a conically shaped portion having a radius gradually decreased toward an outlet is disposed at least on an inner surface at the outlet side of the hollow section.

19. The treatment section operation check instrument according to claim 11, further comprising a shutter member which is retractable with respect to the hollow section of the chamber to form the hollow section with a volume allowing the treatment section to perform a treatment operation when the shutter member is not protruded into the hollow section, and to communicate the pair of communication sections when the shutter member is protruded into the hollow section.

20. A treatment section operation check method comprising steps of:
leading out a treatment section of an endoscopic treatment instrument to a distal end side of the cartridge manually or with a drive force; and
operating the treatment section led out to the distal end side of the cartridge through sliding or the drive force.

21. The treatment section operation check method according to claim 20, further comprising a step of prompting a next operation when the treatment section is operated as specified.

22. A treatment section operation check method of an endoscopic system including an endoscope including a treatment instrument channel, a cartridge which stores a treatment section introduced into the treatment instrument channel and a treatment instrument insertion portion, a control unit with a control section including a memory which registers a treatment section operation check program for performing an operation check of the treatment section in accordance with a type of the treatment instrument, a treatment instrument insertion portion electric driver for driving back and forth the treatment section insertion portion of the treatment instrument under a control of the control unit, a treatment section operation unit for operating the treatment section of the treatment section under the control of the control unit, and a treatment section operation unit with an operation check command button for outputting a command signal to the control unit to perform a check by operating the treatment instrument insertion portion electric driver and the treatment section operation unit based on the treatment section operation check program registered in the memory, the treatment section operation check method comprising steps of:
driving the treatment instrument insertion portion electric driver for bringing at least the treatment section of the endoscopic treatment instrument stored in a sheath storage portion of the cartridge into a hollow section of a chamber of a treatment section operation check instrument from a distal end side of the cartridge;
stopping the drive of the treatment instrument insertion portion electric driver when the treatment section is disposed in the hollow section of the chamber;
driving the treatment section operation unit for operating the treatment section disposed in the hollow section of the chamber;
detecting whether or not the treatment section disposed in the hollow section is operated as specified; and
prompting a next operation after stopping the operation of the treatment section operation unit when the treatment section is operated as specified.

23. The treatment section operation check method of the endoscopic system according to claim 22, further comprising a step of notifying an operator and a staff of an error when the treatment section is not operated as specified.
